(19) United States
(12) Patent Application Publication
(10) Pub. No.: US 2009/0281395 A1
(43) Pub. Date: Nov. 12, 2009

(54) Method of determining the slow wave of a gastrointestinal tract

(76) Inventors: John R. Semler, Williamsville, NY (US); Paul E. Buckley, Elma, NY (US); Andreas J.P.M. Smout, Utrecht (NL)

Correspondence Address: PHILLIPS LYTLE LLP;INTELLECTUAL PROPERTY GROUP, 3400 HSBC CENTER, BUFFALO, NY, 14203-3509, US

(21) Appl. No.: 12/387,609

(22) Filed: May 5, 2009

Related U.S. Application Data
(60) Provisional application No. 61/126,789, filed on May 7, 2008.

(51) Int. Cl. A61B 5/07; A61B 5/00

(52) U.S. Cl. 600/301; 600/302

(57) ABSTRACT

A computerized method of determining the slow wave of a gastrointestinal tract comprising the steps of providing an ingestible capsule (20) having a pressure sensor (29), having a subject ingest the capsule, recording measurements from the pressure sensor as the capsule passes through the gastrointestinal tract of the subject, transmitting the measurements to a processor (31), conditioning the measurements (42) to provide pressure data as a function of a time interval (60), interpolating missing pressure data in the time interval (43), filtering the pressure data as a function of a desired bandpass (44), differencing the pressure data (45), windowing the pressure data (46), applying a sample size and an overlap between samples to segment the pressure data (47), applying a Fourier transform to the segmented pressure data to provide frequency pressure data (48), selecting an FFT frequency bandpass (49), computing power spectrum density of the transformed pressure data for the FFT bandpass (50), and plotting the transformed pressure data (53), whereby a dominate pressure frequency (70) correlating to a slow wave of at least a portion of the gastrointestinal tract is provided.

### CROSS-REFERENCE TO RELATED APPLICATIONS

This application claims the benefit of U.S. Provisional Patent Application No. 61/126,789, filed May 7, 2008. The entire content of such application is incorporated by reference herein.

### TECHNICAL FIELD

The present invention relates generally to ingestible capsules and, more particularly, to a process for determining the slow wave of a gastrointestinal tract.

### BACKGROUND ART

Ingestible capsules are well-known in the prior art. Such capsules are generally small pill-like devices that can be ingested or swallowed by a patient. It is known that such capsules may include one or more sensors for determining physiological parameters of the gastrointestinal tract, such as sensors for detecting temperature, pH and pressure.

A number of methods of determining location of an ingestible capsule are known in the prior art. For example, it is known that signal strength or signal triangulation may be used to attempt to determine the location of an ingested capsule. However, the use of an RF signal has a number of disadvantages, including that it generally requires multiple antennas, various tissues may impact the signal differently, and patient movement may skew the results. It is also known that accelerometers may be used to attempt to determine location, but such methods also have disadvantages, such as drift, non-linear progression and rotational inaccuracy.

It is also known that certain physiological parameters may be associated with regions of the gastrointestinal tract. For example, a 1988 article entitled “Measurement of Gastrointestinal pH Profiles in Normal Ambulant Human Subjects” discloses pH measurements recorded by a capsule passing through the gastrointestinal tract. It is known that pH has been correlated with transitions from the stomach to the small bowel (gastric emptying) and from the distal small bowel to the colon (ileo-caecal transition).

It is known that electrical activity controls the contractions of the stomach and that gastric myoelectrical activity comprises a slow wave (or electrical control activity) and spikes (or electrical response activity). Electrogastrography (EGG) may be used to record gastric electrical activity by placing cutaneous electrodes on the abdomen over the stomach. The dominant frequency of the EGG represents the frequency of the gastric slow wave and spikes are reflected in the EGG as an increase in amplitude. Because direct visual interpretation of the EGG time signal is difficult, quantitative analysis of the EGG using running spectral analysis (or time-frequency representation) may be used. Such methods include short form Fourier transform, adaptive spectral analysis and exponential distribution.

### DISCLOSURE OF THE INVENTION

With parenthetical reference to corresponding parts, portions or surfaces of the disclosed embodiment, merely for the purposes of illustration and not by way of limitation, the present invention provides a computerized method of determining the slow wave of a gastrointestinal tract comprising the steps of providing an ingestible capsule (20) having a pressure sensor (29), having a subject ingest the capsule, recording measurements from the pressure sensor as the capsule passes through the gastrointestinal tract of the subject, transmitting the measurements to a processor (31), conditioning the measurements (42) to provide pressure data as a function of a time interval (60), interpolating missing pressure data in the time interval (43), filtering the pressure data as a function of a desired bandpass (44), differencing the pressure data (45), windowing the pressure data (46), applying a sample size and an overlap between samples to segment the pressure data (47), applying a Fourier transform to the segmented pressure data to provide frequency pressure data (48), selecting an FFT frequency bandpass (49), computing power spectrum density of the transformed pressure data for the FFT bandpass (50), and plotting the transformed pressure data (53), whereby a dominate pressure frequency (70) correlating to a slow wave of at least a portion of the gastrointestinal tract is shown.

The step of transmitting the measurements to a processor may comprise the steps of transmitting the measurements from the capsule to a receiver (17) and downloading the measurements from the receiver to the processor. The step of conditioning the measurements to provide pressure data as a function of a time interval may comprise the steps of screening the measurements to verify that they are valid, converting the measurements to units of pressure, compensating for temperature, and applying a baseline compensation. The missing pressure data may be the result of an error in the pressure sensor measurement (102) or a change in a sampling rate of the sensor (100). The step of interpolating missing pressure data in the time interval may comprise identifying a data gap, fitting a curve to data on each side of the gap, and computing a value for the missing data as a function of the curve. The step of interpolating missing pressure data in the time interval may comprise identifying a data gap, identifying a data value on one side of the gap, and providing a value for the missing data based on the identified data value. The step of filtering the pressure data as a function of a desired bandpass may comprise the step of applying a Butterworth bandpass filter (105) or applying a Butterworth lowpass filter. The step of differencing the pressure data may comprise the step of selecting each data value in a sequence and subtracting the next data value in the sequence from the selected data value. The step of windowing the pressure data may comprise the step of inputting (113) parameters for the windowing. The sample size (110) may be about twenty minutes and the overlap (111) may be about eighteen minutes. The FFT frequency bandpass (114) may be from about 4 CPM to about 15 CPM. The step of computing power spectrum density may comprise the steps of selecting a central frequency and determining an amplitude value for pressure data in the selected frequency, squaring and taking the sum of the amplitudes and amplitude values for pressure data in a number N of neighboring frequencies (109), and repeating the foregoing for each frequency in the FFT frequency bandpass, and N may be about 6. The plot may be a graph, and the graph may further comprise color representing the power spectrum density. The method may further comprise the step of identifying the location of the capsule as a function of the dominant pressure frequency (79). The location may be the small bowel of the gastrointestinal tract or the ileo-caecal junction of the gastrointestinal tract. The step of identifying the location of the capsule may be a function of pH measurements taken by the capsule. The step of identifying the location of the capsule may comprise the steps of providing a pH sensor (22) on the capsule, recording measurements from the pH sensor as the capsule passes through the gastrointestinal tract of the subject, transmitting the measurements to the processor, and providing pH data as a function of a time interval (61). The step of identifying the location of the capsule may further comprise the step of analyzing the pH data relative to a pH reference pattern (65).

The invention also provides a computerized method of determining the slow wave of a gastrointestinal tract comprising the steps of providing an ingestible capsule having a pressure sensor, having a subject ingest the capsule, recording measurements from the pressure sensor as the capsule passes through the gastrointestinal tract of the subject, transmitting the measurements to a processor, conditioning the measurements to provide pressure data as a function of a time interval, applying a sample size and an overlap between samples to segment the pressure data, applying a Fourier transform to the segmented pressure data to provide frequency pressure data, and plotting the transformed pressure data, whereby a dominate pressure frequency correlating to a slow wave of at least a portion of the gastrointestinal tract is shown.

The invention also provides a computer-readable medium having computer-executable instructions for performing a method comprising receive pressure measurements recorded by a pressure sensor on an ingestible capsule ingested by a subject, conditioning the measurements to provide pressure data as a function of a time interval, applying a sample size and an overlap between samples to segment the pressure data, applying a Fourier transform to the segmented pressure data to provide frequency pressure data, and plotting the transformed pressure data, whereby a dominate pressure frequency correlating to a slow wave of at least a portion of the gastrointestinal tract is shown

The invention also provides a system for identifying the slow wave of a gastrointestinal tract comprising an ingestible capsule having a pressure sensor adapted to record pressure data as a function of time as the capsule passes through at least a portion of a subject's gastrointestinal tract, a receiver adapted to received the data when transmitted from the capsule, a processor adapted to communicate with the receiver, a display (32) in communication with the processor, the processor programmed to receive pressure measurements recorded by the pressure sensor, condition the measurements to provide pressure data as a function of a time interval, apply a sample size and an overlap between samples to segment the pressure data, apply a Fourier transform to the segmented pressure data to provide frequency pressure data, and plot the transformed pressure data on the display, whereby a dominate pressure frequency correlating to a slow wave of at least a portion of the gastrointestinal tract is shown.

Accordingly, the general object is to provide a method for determining the slow wave of a gastrointestinal tract from pressure measurements.

Another object is to provide a method for determining the location of an ingested capsule.

These and other objects and advantages will become apparent from the foregoing and ongoing written specification, the drawings, and the claims.

### BRIEF DESCRIPTION OF THE DRAWINGS

FIG. 1 is a sectional view of an ingestible capsule adapted to record pressure and pH measurements in a gastrointestinal tract.

FIG. 2 is a schematic of an embodiment of the improved system.

FIG. 3 is a flow chart of an embodiment of the improved method.

FIG. 4 is a view of a user interface for the embodiment shown in FIG. 3.

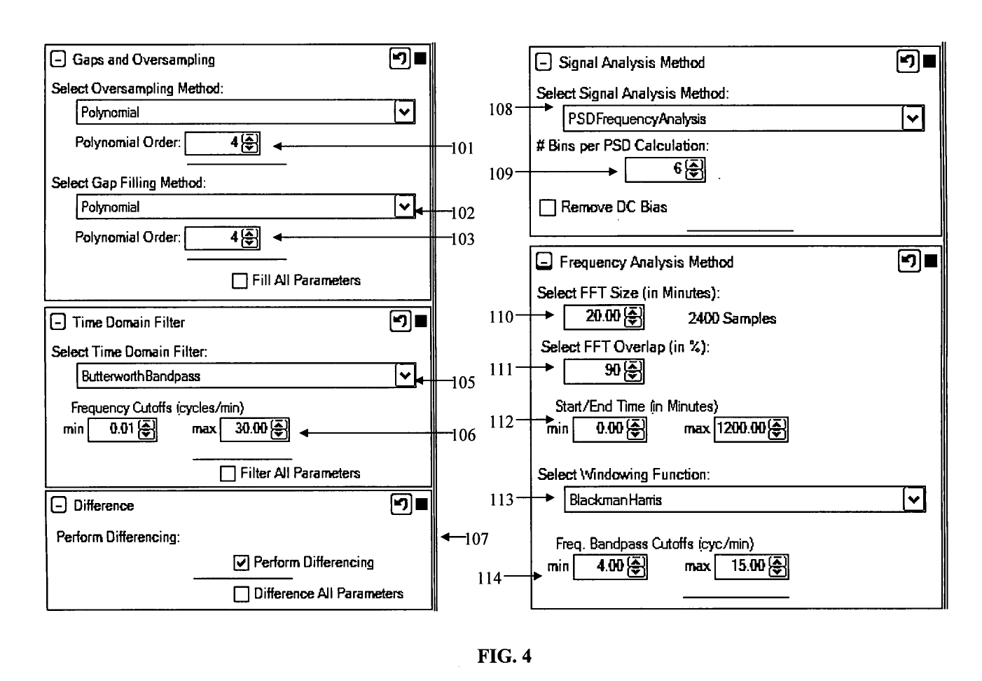

FIG. 5A is a graphical display of pressure date.

FIG. 5B is a graphical display of pressure data shown in FIG. 5A transformed to frequency data using an embodiment of the improved system and method.

FIG. 6 is a prior art graphical view of pH readings taken by a radio telemetry capsule passing through the gastrointestinal tract and also shows various segments of the gastrointestinal tract.

### DESCRIPTION OF PREFERRED EMBODIMENTS

At the outset, it should be clearly understood that like reference numerals are intended to identify the same structural elements, portions or surfaces consistently throughout the several drawing figures, as such elements, portions or surfaces may be further described or explained by the entire written specification, of which this detailed description is an integral part. Unless otherwise indicated, the drawings are intended to be read (e.g., cross-hatching, arrangement of parts, proportion, degree, etc.) together with the specification, and are to be considered a portion of the entire written description of this invention. As used in the following description, the terms “horizontal”, “vertical”, “left”, “right”, “up” and “down”, as well as adjectival and adverbial derivatives thereof (e.g., “horizontally”, “rightwardly”, “upwardly”, etc.), simply refer to the orientation of the illustrated structure as the particular drawing figure faces the reader. Similarly, the terms “inwardly” and “outwardly” generally refer to the orientation of a surface relative to its axis of elongation, or axis of rotation, as appropriate.

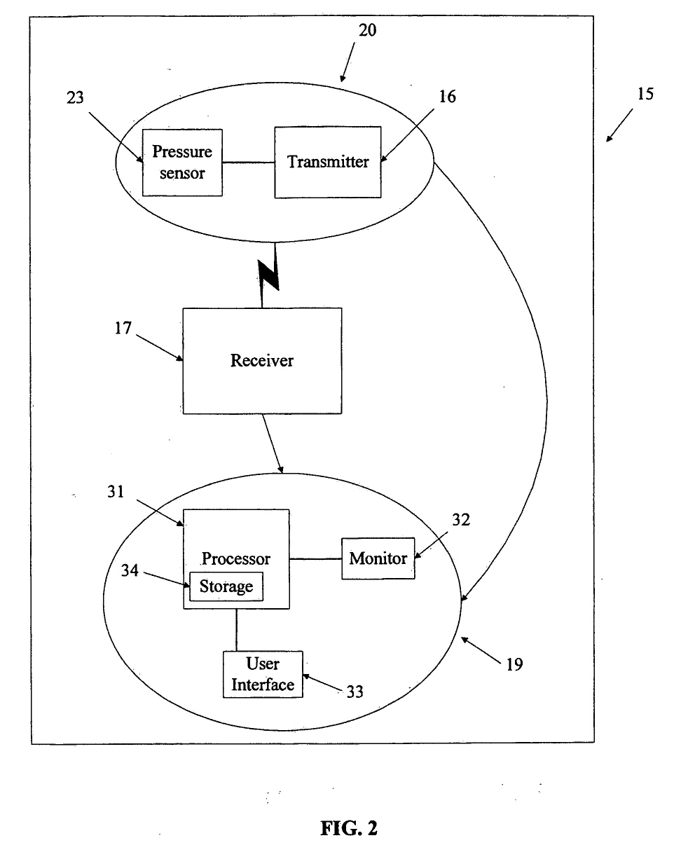

Referring now to the drawings and, more particularly, to FIG. 2 thereof, this invention provides a new system for determining the slow wave of a gastrointestinal tract, of which the presently preferred embodiment is generally indicated at 15. As shown in FIG. 2, system 15 generally includes an ingestible capsule 20 having a pressure sensor assembly 23 for taking measurements of a subject's gastrointestinal tract and a transmitter 16 for transmitting the measurements, a receiver 17 for receiving signals sent from transmitter 16, and a computer workstation 19 for processing measurements from pressure sensor 23 to determine the dominant frequency of the pressure measurements.

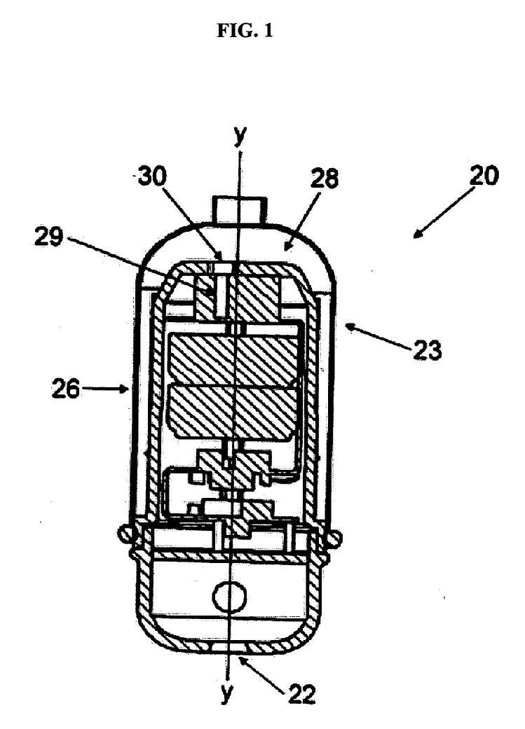

As shown in FIG. 1, capsule 20 is generally a cylindrical member elongated about axis y-y and having generally rounded closed ends, somewhat resembling a medicament capsule. The capsule generally has a hard shell or casing which houses the transmitting electronics, battery compartment and sensors. Capsule 20 is adapted to be ingested or otherwise positioned within a tract to sense pressure, pH and temperature within the tract and to transmit such readings to receiver 17. The capsule is generally provided with an outer surface to facilitate easy swallowing of the capsule. In this embodiment, capsule 20 is an autonomous swallowable capsule and is self-contained. Thus, capsule 20 does not require any wires or cables to, for example, receive power or transmit information. The pH, pressure and temperature data are transmitted from capsule 20 within the gastrointestinal tract to a remote data receiver 17.

Capsule 20 includes a pressure sensor assembly 23 comprising a flexible sleeve 26 affixed to the shell of the capsule and defining a chamber 28 between the shell and the sleeve. Chamber 28 is filled with a fluid, which is a non-compressible medium that transfers a force acting upon sleeve 26 to sensing mechanism 29 of sensor 23. In this embodiment, the fluid used is a dielectric gel. Alternatively, it is contemplated that other fluids, such as mineral oil or an inert gas, may be used. A pressure sensor 29 is operatively arranged to sense pressure within chamber 28 and communicates with the chamber through a fluid port 30 at one end of the shell of the capsule. As shown, the pressure sleeve 26 of capsule 20 extends from a point below the middle of the capsule up over the top end of the capsule. An analog to digital converter is provided to convert the analog signal from sensor 29 to a digital signal.

On the opposite end of capsule 20 to pressure sensor 23 is pH sensor 22. In the preferred embodiment, pH sensor 22 is a conventional ISFET type pH sensor. ISFET stands for ion-selective field effect transistor and the sensor is derived from MOSFET technology (metal oxide screen field effect transistor). A current between a source and a drain is controlled by a gate voltage. The gate is composed of a special chemical layer which is sensitive to free hydrogen ions (pH). Versions of this layer have been developed using aluminum oxide, silicon nitride and titanium oxide. Free hydrogen ions influence the voltage between the gate and the source. The effect on the drain current is based solely on electrostatic effects, so the hydrogen ions do not need to migrate through the pH sensitive layer. This allows equilibrium, and thus pH measurement, to be achieved in a matter of seconds. The sensor is an entirely solid state sensor, unlike glass bulb sensors which require a bulb filled with buffer solution. Only the gate surface is exposed to the sample.

After activation and ingestion, capsule 20 senses and transmits measurements for at least 120 hours after activation. In the preferred embodiment, the range and accuracy of the sensors are generally 0.05 to 9.0 pH units with an accuracy of ±0.5 pH units, 0 to 350 mmHg with an accuracy of 5 mmHg, or 10% above 100 mmHg, and 25° to 49° C. with an accuracy of ±1° C.

In the preferred embodiment, the capsule transmits measurements at about 434 MHz and measures 26.8 mm long by 11.7 mm in diameter. As shown in FIG. 2, portable data receiver 17 worn by the subject receives and stores measurements transmitted by transmitter 16 in capsule 20. Data receiver 17 contains rechargeable batteries and when seated in a docking station allows for battery charging and data download. Data is downloaded from data receiver 17 through a docking station via a USB connection to, in this embodiment, a Windows PC compatible computer 19, such as a conventional laptop or a desktop.

Thus, capsule 20 is provided to a subject and is ingested by the subject. Pressure measurements are recorded 42 by sensor 23 as the capsule passes through at least a portion of the gastrointestinal tract of the subject. The raw data measurements are then transmitted in data packets by transmitter 16 to receiver 17, which is generally worn on the belt of the user outside the gastrointestinal tract of the subject. After the recording period is complete, the receiver is then seated in the docking station, which is connected to computer 19 through a USB connection. The raw data is then transferred from receiver 17 to computer 19. The data is then analyzed by computer 19 and used to make a determination regarding the dominant frequency 79, as further described below.

In this embodiment, computer 19 includes a processor 31, data processing storage 34, a monitor or display 32 and a user input device 33. In this embodiment, monitor 32 is a computer screen. However, monitor 32 may be any other device capable of displaying an image or other data. In this embodiment, user input device 33 includes a keyboard and a mouse. However, user input device 33 could be any other suitable input-output device for interfacing with data processor 31.

The processing and analysis of the pressure measurements from capsule 20 is generally provided using computer-executable instructions executed by a general-propose computer, such as a server or personal computer 19. However, it should be noted that this processing and analysis may be practiced with other computer system configurations, including internet appliances, hand-held devices, wearable computers, multi-processor systems, programmable consumer electronics, network PCs, mainframe computers and the like. The system can be embodied in any form of computer-readable medium or a special purpose computer or data processor that is programmed, configured or constructed to perform the subject instructions. The term computer or processor as used herein refers to any of the above devices as well as any other data processor. Some examples of processors are microprocessors, microcontrollers, CPUs, PICs, PLCs, PCs or microcomputers. A computer-readable medium comprises a medium configured to store or transport computer readable code, or in which computer readable code may be embedded. Some examples of computer-readable medium are CD-ROM disks, ROM cards, floppy disks, flash ROMS, RAM, nonvolatile ROM, magnetic tapes, computer hard drives, conventional hard disks, and servers on a network. The computer systems described above are for purposes of example only. An embodiment of the invention may be implemented in any type of computer system or programming or processing environment. In addition, it is meant to encompass processing that is performed in a distributed computing environment, were tasks or modules are performed by more than one processing device or by remote processing devices that are run through a communications network, such as a local area network, a wide area network or the internet. Thus, the term processor is to be interpreted expansively.

Processor 19 is programmed to extract information from the pressure measurements taken by pressure sensor 23 and to transform it into useable and recognizable forms that can be used to determine the location of the capsule within the gastrointestinal tract and/or assess the health of the subject. In particular, pressure sensor 23 measurements are quantitatively analyzed using running spectrum analysis to determine the dominant frequency 79, which can in turn be correlated to the known slow wave of a portion of the gastrointestinal tract.

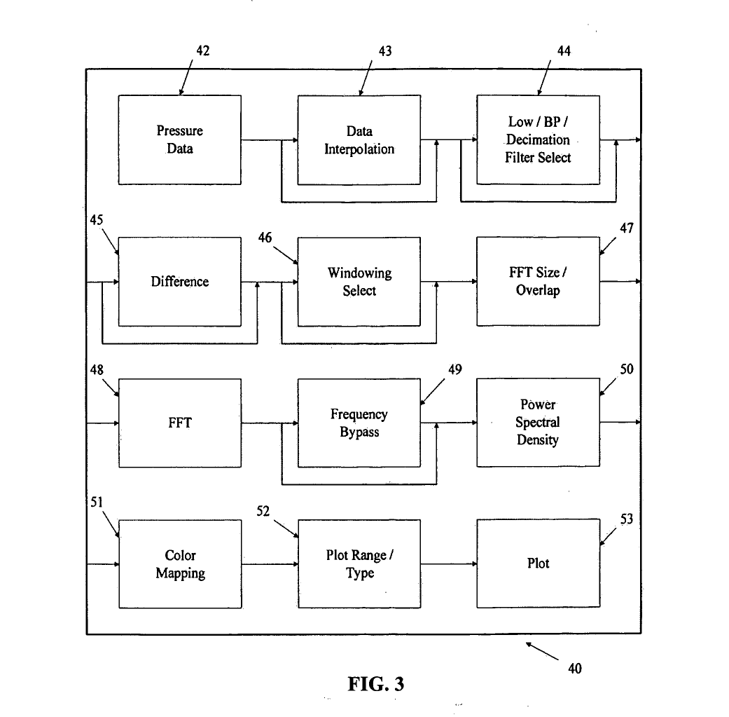

As shown in FIG. 3, pressure measurements are processed 40 in a series of steps and in a manner that allows the user to select different parameters for the processing. The raw measurements from pressure sensor 23 are conditioned 42 to provide pressure data 60 as a function of a time interval. The measurements, which are transmitted from capsule 20 in packets, are screened to verify that they are valid and have not been corrupted using a conventional packet validation process. The measurements are also converted to units of pressure, which are pascals (Pa) or millimeters of mercury (mmHg) in this embodiment. The measurements are also compensated for temperature, as the measurement values may need to be adjusted for temperature variations from standard in the gastrointestinal tract.

In this embodiment, a number of data structures are employed. In this embodiment, the data sets are converted to real, double precision, number arrays. The conditioned time-domain data, which in addition to pressure may include in the preferred embodiment pH and temperature, is organized into an array of values or data sets for time domain functions. Besides the time-domain data sets, a set of double-precision arrays are employed to hold segmented time-domain data. The time-domain data set is sliced into fixed-length segments and each segment overlaps the next by some amount. As described below, in the preferred embodiment this overlap is ninety percent (90%). A set of arrays are used to hold FFT transformed frequency data. This array is a function of the FFT type and the length of the time-domain data that is transformed. Typically the size is twice the length of the time-domain data.

Once a time-domain data array is created, missing pressure data in the time interval is interpolated 43. Missing data or gaps may occur for two reasons. Gaps in the data may arise from a malfunction or error in pressure sensor 23. These gaps can be filled in four different ways, and system 15 allows the user to select 102 from one of these options. The first option is to simply zero all values in the gap. The second option is to repeat the previous sample in the sequence or use the values from the previous sample. The third option is to linearly interpolate between N and N+1 samples. The data gap is identified and a curve is fit to the data values on each side of the data gap. Values for the missing data are then computed as a function of the curve. Alternatively, a polynomial interpolation between N and N+1 samples using a P-order 103 curve fit among N points before and after the N sample may be applied. A 4th-order curve fit is recommended in the preferred embodiment. However, if the gap occurs near the beginning or the end of the data set a 4th-order curve fit may not be possible.

Missing data may also arise from differences in the sampling rate of the pressure sensor during the subject time interval as the capsule moves through gastrointestinal tract. In the preferred embodiment, pressure sensor 23 takes two samples per second (samples at 2 Hz) during the first 24 hours. After the first 24 hours, the pressure sensor takes readings once per second (samples at 1 Hz). Due to this methodology, the data may go from a 2 Hz to a 1 Hz data rate. A number of different over-sampling methods 100 may be used to get all the data to a 2 Hz data rate. The first option is to repeat the previous sample. The second option is to linearly interpolate between N and N+1 samples. The third option is two apply a polynomial interpolation between N and N+1 samples using a P-order 10/curve fit among N points before and after the N sample. Again, a 4th order curve fit is recommended, unless the gap is near the very beginning or very end of the data set. Near the very beginning or the very end of the data set it may be preferable to simply repeat the first available datum backwards to the beginning to the data set or the last available datum forward to the end of the data set.

Thus, for each input data set, all gaps are filled and over-sampling is applied, if necessary, to get the data to a 2 Hz data rate. The data is stored with the appropriate time-domain array type, and is marked as available for subsequent use. The data arrays length (number of elements in the resulting data set) is saved. The number of converted data sets in the target array is also tracked.

The data is then filtered as a function of a desired bandpass 44. A conventional lowpass or bandpass filter may be selected 105. Depending on its impletion, this filtering function may change (shorten) the length of data set and may also shift the data in time, typically by one-half the filter length. In the preferred embodiment, a conventional two or three-stage Butterworth filter or a conventional Butterworth lowpass filter may be used. Alternatively, the user may select to not apply any filter. The user is also able to select 106 the high and low cutoff frequencies. For the lowpass filter, a low cutoff frequency must be specified. The high frequency cutoff should be less than one-half the sampling frequency (1 Hz in the preferred embodiment) or else aliasing artifacts may appear in the resulting data set. The low frequency cutoff should generally be less than the lowest expected gastrointestinal contraction, although this is not required. Of course, the low frequency cutoff must be less than the high frequency cutoff. The user may also choose the data set to which the filter should be applied. The output from this filtering step is data sets in the same format as the previous step. If no filtering is selected the data is simply copied from the input data sets to the output data sets.

Next, differencing 45 is applied to the pressure data. In the preferred embodiment, the difference function subtracts every N+1 data value from the Nth data value. Thus, each data value in the sequence is selected and the next data value in the sequence is subtracted from the selected data value. The length of the output data set will therefore be one less than the length of the input data set. This differencing assists in exaggerating sudden changes in value, so for relatively smooth pressure data sets the resulting data set will create peaks at both ends of each contraction. In the frequency domain this should show up as an exaggerated spike at the dominant frequency. However, the user may select 107 not to difference the data or may even choose the data set to which the differencing should be applied. The output from differencing are the data sets in the same format as a previous process step, although the data set length may change as mentioned above.

Next, FFT windowing is applied 46. A number of conventional FFT windowing functions may be used. These window functions help depress frequency bleed (or smearing), into nearby frequency bins. The windowing function is applied to each data subset to be transformed and its purpose is to de-weight the ends of the data sets so that the first and last values are equal (usually zero). The user may select 113 from a number of different windowing options. These options including, from the most narrowing function to the least narrowing function, a conventional Blackman-Harris function (in this function an additional parameter may be the number of terms, which can range from four to seven), a conventional Kaiser-Bessel function, a conventional Flat-Top function, a conventional Hamming function, and conventional Hanning function, or a Rectangular function (comparable to no windowing).

A sample size and overlap between samples to be segmented is then selected for the FFT analysis 47. In the preferred embodiment, the FFT size is selected 110 in minutes. Thus, the user is able to select an FFT sample size, usually a power of two. However, as conventional FFTs can now process sample sizes in the form of 2^{M}3^{N}5^{P}, it is contemplated that the user may select any sample size in minutes, and then adjust the result the closest to 2^{M}3^{N}5^{P} if necessary. The table below shows representative results of this approach.

| Sample size (minutes) | Closest 2^{M}3^{N} | Adjusted size (minutes) |
|---|---|---|
| 5 | 2³ * 3¹ 5² (600) | 5.0 |
| 10 | 2⁴ * 3¹ * 5² (1200) | 10.0 |
| 13 | 2⁹ * 3¹ (1536) | 12.8 |
| 15 | 2³ * 3² * 5² (1800) | 15.0 |
| 20 | 2⁵ * 3¹ * 5² (2400) | 20.0 |
| 25 | 2³ * 3¹ * 5³ (3000) | 25.0 |
| 30 | 2⁴ * 3² * 5² (3600) | 30.0 |

In the preferred embodiment, the user selects an overlap between segments as a percentage 111 to minimize graphical discontinuities. If the overlap is designated in minutes rather than a percent overlap, the program should take into account the overlap based on the unadjusted sample length (not the adjusted length), and then compute the percent overlap. For example, if the user selects a 13 minute sample length with a 6.5 minute overlap, the percent overlap is fifty percent or 6.4 minutes of 1536 samples on an adjusted sample length of 12.8 minutes. In the preferred embodiment, a sample length of 20 minutes is preferred. In addition, an overlap of ninety percent is preferred. The user is also provided an opportunity to select 112 the overall time range over which the FFT will be applied. The output of step 45 is a list of segment arrays, with each array having the selected windowing function 46 applied to it.

A conventional fast Fourier transform (FFT) 48 is applied to the segments to provide frequency pressure data. The segment data arrays are input to the FFT and the FFT output are FFT arrays. In the preferred embodiment, the total FFT frequency range is fixed at 120 cycles per minute (CPM), which is derived from the original 2 Hz sample rate. The first frequency bin is DC (0 CPM). Each frequency will have a bandwidth of 120 CPM per array length. The dominant frequency of the pressure data 60 may be identified when the FFT transformed frequency data is plotted as further described below. The dominant frequency is the frequency, at any moment in time, exhibiting the most power. Once the dominant frequency 79 has been teased out of the pressure measurements, it is compared to the known slow wave of different portions of the gastro-intestinal tract to determine the location of the capsule and/or the health of the subject.

The user may also select 114 a frequency range of interest or a frequency bandpass 49. The bandpass applied in the preferred embodiment is about 4 CPM to about 15 CPM. Outside this selected range, the FFT data values are set to zero. Alternatively, the user may elect not to apply a bandpass, which in the preferred embodiment is equivalent to a low value of zero and a high value of 60 CPM.

The user may also select 108 to compute power spectral density (PSD) 50. In the preferred embodiment, the PSD is computed by taking the sum of the squares of all amplitudes in a central bin plus N of its neighbors. This process is repeated for all bins or frequencies within the bandpass region. Alternatively, it could be repeated for all bins in the entire FFT array without regard to bandpass, as the bandpass filter will cause zeroing of all data outside the bandpass region in any case. Some care must be taken at the beginnings and ends of each array to compensate for non-existing bins. In these regions, the PSD will be computed only using those bins that actually exist, rendering invalid results for the bins close to 0 CPM and 60 CPM. In the preferred embodiment, the user may select 109 the number of adjacent frequency bins N to include in the calculation. In the preferred embodiment, this number 109 is preferably set at 6. If the number of bins 109 is set at just 1, then it is as if the FFT analysis is conducted without PSD. Alternatively the user could select the frequency width of the PSD, for example 0.5 CPM, and an internal calculation may be used to determine the total number of bins that this width encompasses. The PSD outputs FFT arrays and the values of these arrays are representative of power or energy.

Two options are available for color mapping 51 and plotting 53. The first option is to plot the PSD by coloring the graph with a scale that is same for the entire graph. For example, all amplitudes of value 10 would be designated in red, all amplitude values of 5 would be designated in green, and all amplitude values of 1 would be designated in blue. At any given time, all frequencies of lesser power would be colored down the spectrum all the way to black. Thus, this plot colors red as the maximum power relative to the entire graph, and then colors all other pixels on the graph a lesser color based on the pixel's power relative to the maximum power. This plot is good at revealing when, in time, the most contraction energy is being expended during the digestion process.

Alternatively, a dominant frequency plot may be generated in which the graph plots colors with a relative scale that is different for each point on the time axis. Thus, the highest amplitude for time X would be red and the highest amplitude for time Y would also be red although these amplitudes might be different values. Thus, as the user views the plot at various time points, all the dominant frequencies show up at the same color—bright red. This makes it easy to spot the characteristic dominant frequency curves.

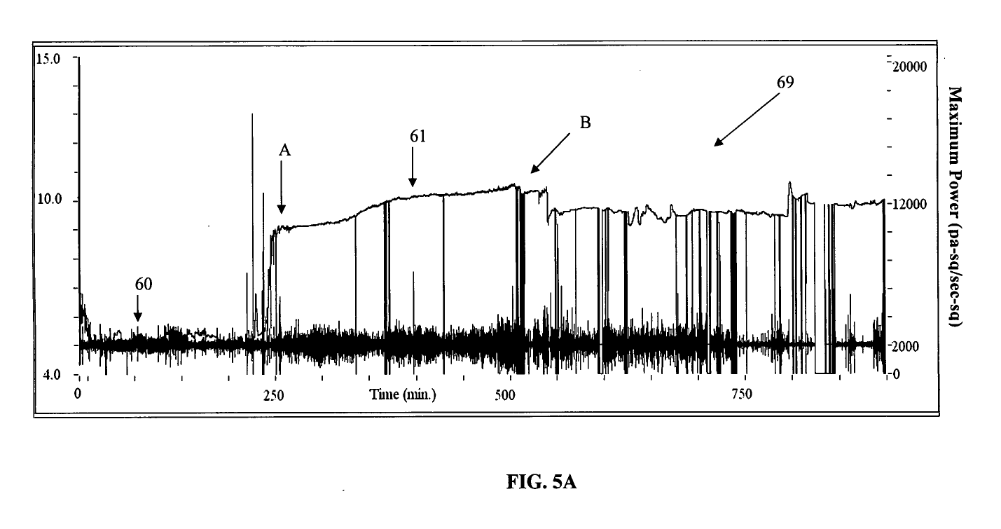

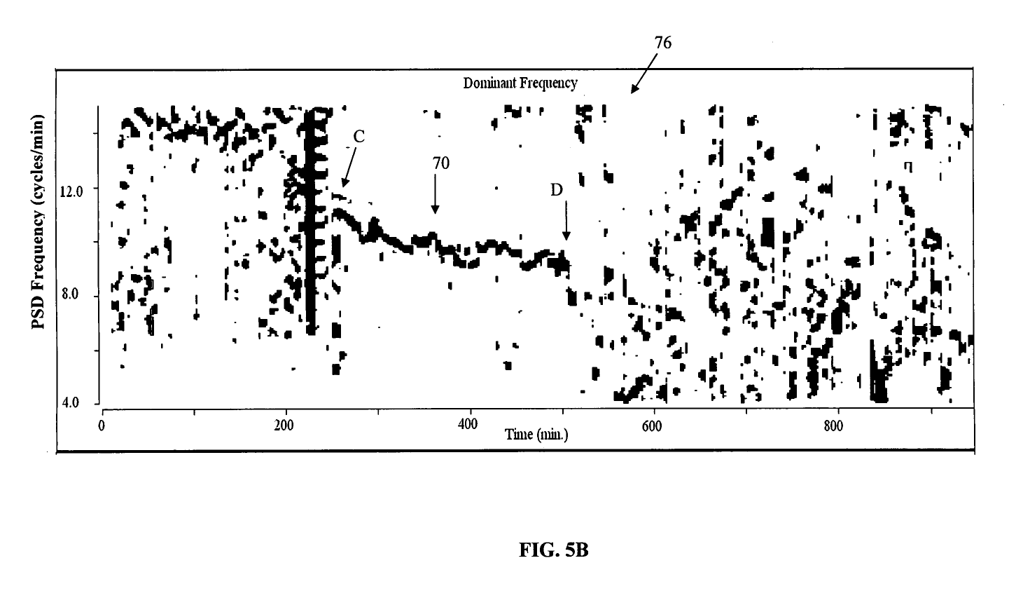

The colors for the amplitudes are selected during the color mapping stage 51. Thus, the output graph shown in FIG. 5B is really a three-dimensional graph. Instead of having amplitude on a Z-axis, however, color is used to represent amplitude. Once colors are selected for the subject amplitudes 51, the user may manipulate the graph display 52. For example, how much of the time interval is shown on the plot and the data to be plotted may be modified, as may be the type of plot. Other characteristics such as brightness and contrast may also be modified. Once the selections are made, the frequency data is plotted and a graph 79 is shown on monitor 32. As shown in FIG. 5A, a graph 69 of pressure data 60, as well as pH data 61, may be plotted for the subject time interval. As shown in FIG. 5B, a second graph 79 of the dominant frequency data may be plotted below for the subject time interval.

A number of the subroutines employed herein may be performed with the use of conventional software tools. For example, Measurement Studio for Visual Studio (Version 8), licensed by National Instruments of 11500 North Mopac Expressway, Houston, Tex. 78759-3504, may be used in the preferred embodiment to perform a number of the steps including time domain filter 44, windowing function 46, FFT size/overlap 47 and to FFT 48. Measurement Studio also provides plotting functionality that may be used in the preferred embodiment.

Using system 40, pressure sensor 23 measurements are quantitatively analyzed using running spectrum analysis to determine the dominant frequency 79, which can in turn be correlated to the known slow wave of a portion of the gastrointestinal tract. Average pressure readings from the capsule plotted against transit time are shown in FIG. 5A. The dominant frequency of those pressure readings plotted against the same overall time period are shown in FIG. 5B. As shown, a slightly downwardly sloped viable line 70 can be seen on plot 79 between C and D, representing a gradual decrease in the dominant frequency 70 between time C and time D. The slow wave of a healthy subject at the start of the small bowel is known to be about 12 CPM and in the ileum is known to be about 7 CPM. Thus, dominant frequency represented by the line 70 between C and D of the pressure data measured by the capsule correlates to the slow wave of the small bowel. The location of the capsule may therefore be determined by a comparison of the dominant frequency and a reference slow wave. Furthermore, if it has already been determined by other methods that the capsule is in the duodenum and the dominant frequency differs substantially from a reference slow wave, then this information may be used to diagnose a health condition or abnormality in the subject. Thus, in the preferred embodiment, dominant frequency patterns derived from pressure measurements taken by the capsule as it passes through the gastrointestinal tract are used to determine the capsule's location and the health of the subject.

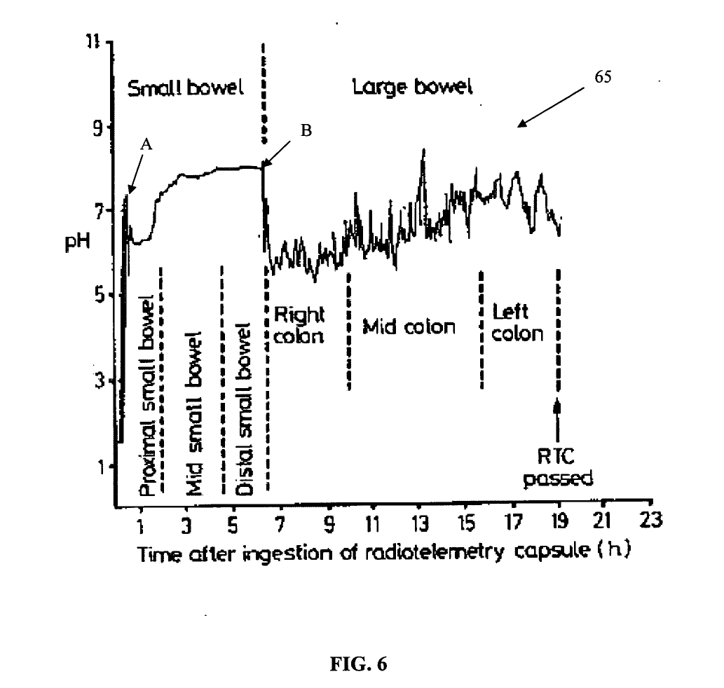

As shown in FIG. 6, based on reference data, a substantial variation or increase in pH, generally indicated at A, indicates passage of the capsule from the stomach to the small intestine, often referred to as gastric emptying. A latter variation in pH, indicated at B, suggests movement of the capsule from the ileum to the caecum. It has been found that this significant pH drop is seen some hours after gastric emptying and is due to the capsule moving from the ileum to the caecum, a transition referred to as the ileo-caecal junction. Intraluminal pH of the gastrointestinal tract drops between the ileum and the more acidic caecum due to formation of bacteria in the colon.

Also shown in FIG. 5 is that the start of the dominant frequency line 70 between C and D was generally found to occur, as indicated at C, at a time corresponding to the gastric emptying A suggested by the graph of pH shown in FIGS. 5A and 6. This correlation between the variation in dominant frequency C and the variation in pH A may also be used as a reference to confirm that the capsule has moved from the stomach to the small bowel. The end of dominant frequency line 70 between C and D occurs, as indicated at D, at a time corresponding to the ileo-caecal junction B suggested by the graph of pH shown in FIGS. 5A and 6. This correlation between the variation in dominant frequency D and the variation in pH B may also be used as a reference to determine that the capsule has moved from the ileum to the caecum of the subject.

In comparing patterns 66, 76 from a subject with reference templates for pH 65 and the slow wave of the gastrointestinal tract, if there is a correlation between pH and a reference pH and a correlation between dominant frequency and a reference slow wave, then a determination of the capsule's location may be more accurate. Without this correlation, the capsule being located at or near the ileo-caecal junction, for example, may be less certain.

After an overnight fast, 56 healthy volunteers (33 male and 23 female with a mean age of 34.4) swallowed a capsule 22 after a standardized meal (containing 120 gram egg beaters, 2 pieces of bread with jam, 255 K cal. and two percent (2%) fat) and 120cc's of water. Approximately 5.5 hours after the gastric expulsion of the capsule, a drop of pH of greater than 1 unit for more than 5 minutes was seen. Contractile frequency was assessed with the described power spectral density analysis of the pressure tracing recorded by the capsule and was analyzed using the above process using a multi-taper method for 2 minute windows shifted every 30 seconds. The frequency at the peak power was found and the mean peak frequency was calculated at 30 minute windows, before and after the beginning of the pH drop. These means were then compared by paired two-tailed T-test. The average time between the gastric emptying and pH drop into the caecum in this sample population was 5.16 hours. The mean dominant frequency of contractions for the 30 minutes before the pH drop was 6.92 CPM. (CI 95%=(6.47-7.36), SD=1.66) and 30 minutes after was 5.82 CPM (CI 95%=(4.96-6.68), SD=3.20), with a significance P<0.02. Thus, significant differences in the mean dominant frequency of contracts occurred around the pH drop, hours after the gastric emptying of the capsule.

With the determination that the capsule has passed from the stomach to the small bowel and then through the ileo-caecal junction, transit time through the small bowel can also be ascertained. Transit time through the colon can then be determined as well. This is useful in a number of clinical applications.

While the preferred embodiment has been described in relation to the gastrointestinal tract of a human, it is contemplated that the system may be used in connection with the gastrointestinal tract of other animals.

The present invention contemplates that many changes and modifications may be made. Therefore, while the presently-preferred form of the improved method has been shown and described, and a number of alternatives discussed, persons skilled in this art will readily appreciate that various additional changes and modifications may be made without departing from the spirit of the invention, as defined and differentiated by the following claims.

What is claimed is:

1. A computerized method of determining the slow wave of a gastrointestinal tract comprising the steps of:
providing an ingestible capsule having a pressure sensor;
having a subject ingest said capsule;
recording measurements from said pressure sensor as said capsule passes through at least a portion of said gastrointestinal tract of said subject;
transmitting said measurements to a processor;
conditioning said measurements to provide pressure data as a function of a time interval;
interpolating missing pressure data in said time interval;
filtering said pressure data as a function of a desired bandpass;
differencing said pressure data;
windowing said pressure data;
applying a sample size and an overlap between samples to segment said pressure data;
applying a Fourier transform to said segmented pressure data to provide frequency pressure data;
selecting an FFT frequency bandpass;
computing power spectrum density of said transformed pressure data for said FFT bandpass;
plotting said transformed pressure data;
whereby a dominate pressure frequency correlating to a slow wave of at least a portion of said gastrointestinal tract is shown.

2. The method set forth in claim 1, wherein said step of transmitting said measurements to a processor comprises the steps of:
transmitting said measurements from said capsule to a receiver outside of said gastrointestinal tract of said subject; and
downloading said measurements from said receiver to said processor.

3. The method set forth in claim 1, wherein said step of conditioning said measurements to provide pressure data as a function of a time interval comprises the steps of:
screening said measurements to verify that they are valid;
converting said measurements to units of pressure;
compensating for temperature; and
applying a baseline compensation.

4. The method set forth in claim 1, wherein said missing pressure data is the result of an error in said pressure sensor measurement or a change in a sampling rate of said sensor.

5. The method set forth in claim 1, wherein said step of interpolating missing pressure data in said time interval comprises:
identifying a data gap;
fitting a curve to data on each side of said gap;
computing a value for said missing data as a function of said curve.

6. The method set forth in claim 1, wherein said step of interpolating missing pressure data in said time interval comprises:
identifying a data gap;
identifying a data value on one side of said gap; and
providing a value for said missing data based on said identified data value.

7. The method set forth in claim 1, wherein said step of filtering said pressure data as a function of a desired bandpass comprises the step of applying a Butterworth bandpass filter or applying a Butterworth lowpass filter.

8. The method set forth in claim 1, wherein said step of differencing said pressure data comprises the step of selecting each data value in a sequence and subtracting the next data value in said sequence from said selected data value.

9. The method set forth in claim 1, wherein said step of windowing said pressure data comprises the step of inputting parameters for said windowing.

10. The method set forth in claim 1, wherein said sample size is about twenty minutes.

11. The method set forth in claim 1, wherein said overlap is about eighteen minutes.

12. The method set forth in claim 1, wherein said FFT frequency bandpass is from about 4 CPM to about 15 CPM.

13. The method set forth in claim 1, wherein said step of computing power spectrum density comprises the steps of:
selecting a central frequency and determining an amplitude value for pressure data in said selected frequency;
squaring and taking the sum of said amplitudes and amplitude values for pressure data in a number N of neighboring frequencies; and
repeating the foregoing for each frequency in said FFT frequency bandpass.

14. The method set forth in claim 13, wherein said N is 6.

15. The method set forth in claim 1, wherein said plot is a graph.

16. The method set forth in claim 15, wherein said graph further comprises color representing said power spectrum density.

17. The method set forth in claim 1, and further comprising the step of identifying the location of said capsule as a function of said dominant pressure frequency.

18. The method set forth in claim 17, wherein said location is the small bowel of said gastrointestinal tract.

19. The method set forth in claim 17, wherein said location is the ileo-caecal junction of said gastrointestinal tract.

20. The method set forth in claim 17, wherein said step of identifying the location of said capsule is a function of pH measurements taken by said capsule.

21. The method set forth in claim 20, wherein said step of identifying the location of said capsule further comprises the steps of:
providing a pH sensor on said capsule;
recording measurements from said pH sensor as said capsule passes through said gastrointestinal tract of said subject;
transmitting said measurements to said processor; and
providing pH data as a function of said time interval.

22. The method set forth in claim 21, and further comprising the step of comparing said pH data to a pH reference.

23. A computerized method of determining the slow wave of a gastrointestinal tract comprising the steps of:
providing an ingestible capsule having a pressure sensor;
having a subject ingest said capsule;
recording measurements from said pressure sensor as said capsule passes through at least a portion of said gastrointestinal tract of said subject;
transmitting said measurements to a processor;
conditioning said measurements to provide pressure data as a function of a time interval;
applying a sample size and an overlap between samples to segment said pressure data;
applying a Fourier transform to said segmented pressure data to provide frequency pressure data;
plotting said transformed pressure data;
whereby a dominate pressure frequency correlating to a slow wave of at least a portion of said gastrointestinal tract is shown.

24. The method set forth in claim 23, wherein said step of transmitting said measurements to a processor comprises the steps of:
transmitting said measurements from said capsule to a receiver outside of said gastrointestinal tract of said subject; and
downloading said measurements from said receiver to said processor.

25. The method set forth in claim 23, wherein said step of conditioning said measurements to provide pressure data as a function of a time interval comprises the steps of:
screening said measurements to verify that they are valid;
converting said measurements to units of pressure;
compensating for temperature; and
applying a baseline compensation.

26. The method set forth in claim 23, wherein said sample size is about twenty minutes.

27. The method set forth in claim 23, wherein said overlap is about eighteen minutes.

28. The method set forth in claim 23, wherein said plot is a graph.

29. The method set forth in claim 23, and further comprising the step of identifying the location of said capsule as a function of said dominant pressure frequency.

30. The method set forth in claim 29, wherein said location is the small bowel of said gastrointestinal tract.

31. The method set forth in claim 29, wherein said location is the ileo-caecal junction of said gastrointestinal tract.

32. A computer-readable medium having computer-executable instructions for performing a method comprising:
receive pressure measurements recorded by a pressure sensor on an ingestible capsule ingested by a subject;
conditioning said measurements to provide pressure data as a function of a time interval;
applying a sample size and an overlap between samples to segment said pressure data;
applying a Fourier transform to said segmented pressure data to provide frequency pressure data;
plotting said transformed pressure data;
whereby a dominate pressure frequency correlating to a slow wave of at least a portion of said gastrointestinal tract is shown.

33. The medium set forth in claim 32, wherein said conditioning said measurements to provide pressure data as a function of a time interval comprises:
screening said measurements to verify that they are valid;
converting said measurements to units of pressure;
compensating for temperature; and
applying a baseline compensation.

34. The medium set forth in claim 32, wherein said sample size is about twenty minutes.

35. The medium set forth in claim 32, wherein said overlap is about eighteen minutes.

36. The medium set forth in claim 32, wherein said plot is a graphical representation of said transformed pressure data.

37. The medium set forth in claim 32, and further comprising identifying the location of said capsule as a function of said dominant pressure frequency.

38. The medium set forth in claim 37, wherein said location is the small bowel of said gastrointestinal tract.

39. The medium set forth in claim 37, wherein said location is the ileo-caecal junction of said gastrointestinal tract.

40. A system for identifying the slow wave of a gastrointestinal tract comprising:
an ingestible capsule having a pressure sensor adapted to record pressure data as a function of time as said capsule passes through at least a portion of a subject's gastrointestinal tract;
a receiver adapted to received said data when transmitted from said capsule;
a processor adapted to communicate with said receiver;
a display in communication with said processor;
said processor programmed to
receive pressure measurements recorded by said pressure sensor,
condition said measurements to provide pressure data as a function of a time interval,
apply a sample size and an overlap between samples to segment said pressure data,
apply a Fourier transform to said segmented pressure data to provide frequency pressure data, and
plot said transformed pressure data on said display;
whereby a dominate pressure frequency correlating to a slow wave of at least a portion of said gastrointestinal tract is shown.

41. The system set forth in claim 40, wherein said conditioning said measurements to provide pressure data as a function of a time interval comprises:
screening said measurements to verify that they are valid;
converting said measurements to units of pressure;
compensating for temperature; and
applying a baseline compensation.

42. The system set forth in claim 40, wherein said sample size is about twenty minutes.

43. The system set forth in claim 40, wherein said overlap is about eighteen minutes.

44. The system set forth in claim 40, wherein said plot is a graphical representation of said transformed pressure data.
